(19) United States
(12) Patent Application Publication
(10) Pub. No.: US 2022/0322999 A1
(43) Pub. Date: Oct. 13, 2022

(54) Systems and Methods for Detecting Sleep Activity

(71) Applicants: Emory University, Atlanta, GA (US); Georgia Tech Research Corporation, Atlanta, GA (US)

(72) Inventors: Gari Clifford, Decatur, GA (US); Ayse Cakmak, Atlanta, GA (US); Christopher Rozell, Decatur, GA (US); Adam Willats, Atlanta, GA (US)

(21) Appl. No.: 17/640,405

(22) PCT Filed: Sep. 4, 2020
(86) PCT No.: PCT/US2020/049392

§ 371 Date: Mar. 4, 2022

Related U.S. Application Data
(60) Provisional application No. 62/896,391, filed on Sep. 5, 2019.

(51) Int. Cl. A61B 5/00; A61B 5/0205; A61B 5/11; G16H 40/67; G16H 50/70

(52) U.S. Cl. CPC A61B 5/4809; A61B 5/4812; A61B 5/0205; A61B 5/1118; A61B 5/7275; G16H 40/67; G16H 50/70; A61B 5/02416

(57) ABSTRACT

The present disclosure relates to systems and methods for detecting sleep-wake activity of a subject using change-point events determined from physiological and/or movement measures. In one implementation, the method may include obtaining at least one set of sensor data generated by one or more sensors for a period of time. The method may also include generating at least two measures from the at least one set of sensor data. The method may further include determining a series of change point events for each measure for the period of time. The method may include determining a sleep stage for each interval of the period of time from at least two sleep stages by processing the series of change point events for each measure using a sleep stage classifier. The sleep stage classifier may include a set of parameters for each measure.

### CROSS-REFERENCE TO RELATED APPLICATIONS

This application claims the benefit of U.S. Provisional Application No. 62/896,391 filed Sep. 5, 2019. The entirety of this application is hereby incorporated by reference for all purposes.

### STATEMENT REGARDING FEDERALLY SPONSORED RESEARCH OR DEVELOPMENT

This invention was made with government support under CCF-1409422 and U.S. Pat. No. 1,636,933 awarded by the National Science Foundation. The government has certain rights in the invention.

### BACKGROUND

Conventional sleep/wake classification techniques (e.g., Oakley algorithm) for wearables are generally based solely on actigraphy derived from accelerometer data. Using only these movement signals can result in incorrect classification of sleep and wake activity. More specifically, these techniques generally overestimate sleep and under-estimate wake. Additionally, there is limited computational power and memory associated with wearables.

### SUMMARY

Thus, there is a need for accurate and efficient detection of sleep-wake activity with minimal computational resources, such as memory.

Techniques disclosed herein relate generally to detecting sleep stages (e.g., sleep-wake activity) of a subject using change-point events determined from physiological and/or movement measures. More specifically, one or more sensors, for example, of a wearable device, may be used to measure sensor data of the subject over a period of time, and one or more physiological and/or movement measures be determined from the sensor data. Each measure may be then be analyzed to determine a set of plurality of change point events. Each set of change-point events may be used to determine the sleep stage of the subject associated with the period of time.

The disclosed embodiments may include computer-implemented systems and methods for determining sleep stage using change point events for one or more measures. The disclosed embodiments may include, for example, a computer-implemented method for determining a sleep stage. The method may be implemented using one or more processors. The method may include receiving or obtaining at least one set of sensor data generated by one or more sensors worn by a subject/user for a period of time. The method may include generating at least two measures from the at least one set of sensor data. The method may further include determining a series of change point events for each measure for the period of time. The method may include determining a sleep stage for each interval of the period of time from at least two sleep stages by processing the series of change point events for each measure using a sleep stage classifier. The sleep stage classifier may include a set of parameters for each measure. The set of parameters for each measure may include one or more coupling parameters. Each coupling parameter may be related to the cross-correlation between the each measure and another one of the measures.

In some embodiments, the one or more sensors and the one or more processors may be of a wearable electronic device. In some embodiments, the one or more sensors may include a photoplethysmographic (PPG) sensor and an accelerometer.

In some embodiments, the at least two measures may include actigraphy, tilt angle, and heart rate. The determining the at least two measures may include determining the heart rate from the sensor data from the PPG sensor and determining the actigraphy and the tilt angle from the sensor data from the accelerometer.

In some embodiments, the one or more sleep stages may include a sleep stage and a wake stage. The set of parameters for each measure may include a sleep stage change event parameter and a history parameter.

In some embodiments, the measures may include three measures. The set of parameters for each measure may include two coupling parameters.

In some embodiments, the determining one or more sleep stages for each interval of the period of time may include applying the set of parameters for each measure to respective series of change point events to determine a probability of a change event; and determining a probability of a change event for each interval of the period of time using each probability for each measure.

In some embodiments, the determining one or more sleep stages for each interval of the period of time may include determining a sleep stage likelihood for each interval using the probability of the change event for each interval of time of the period of time and determining the sleep stage for each interval of time of the period of time from the sleep stage likelihood.

In some embodiments, the method may further include determining sleep information using the sleep stage for each interval of the period of time.

The disclosed embodiments may also include, for example, a system for determining a sleep stage. The system may include a wearable electronic device to be worn by a use. The wearable electronic device may include one or more sensors configured to generate sensor data. The system may further include one or more processors; and a non-transitory machine readable storage medium storing computer-executable instructions which, when executed by the one or more processors, cause the one or more processors to obtain at least one set of sensor data generated by one or more sensors for a period of time. The instructions may further cause generating at least two measures from the at least one set of sensor data. The instructions may also cause determining a series of change point events for each measure for the period of time; and determining a sleep stage for each interval of the period of time from at least two sleep stages by processing the series of change point events for each measure using a sleep stage classifier. The sleep stage classifier may include a set of parameters for each measure. The set of parameters for each measure may include one or more coupling parameters. Each coupling parameter may be related to the cross-correlation between the each measure and another one of the measures.

In some embodiments, the one or more sensors may include a photoplethysmographic (PPG) sensor and an accelerometer.

In some embodiments, the at least two measures may include actigraphy, tilt angle, and heart rate. The determining the at least two measures may include determining the heart rate from the sensor data from the PPG sensor and determining the actigraphy and the tilt angle from the sensor data from the accelerometer.

In some embodiments, the one or more sleep stages may include a sleep stage and a wake stage. The set of parameters for each measure may include a sleep stage change event parameter and a history parameter.

In some embodiments, the measures may include three measures. The set of parameters for each measure may include two coupling parameters.

In some embodiments, the determining one or more sleep stages for each interval of the period of time may include applying the set of parameters for each measure to respective series of change point events to determine a probability of a change event; and determining a probability of a change event for each interval of the period of time using each probability for each measure.

In some embodiments, the determining one or more sleep stages for each interval of the period of time may include determining a sleep stage likelihood for each interval using the probability of the change event for each interval of time of the period of time and determining the sleep stage for each interval of time of the period of time from the sleep stage likelihood.

In some embodiments, the instructions may further cause determining sleep information using the sleep stage for each interval of the period of time.

In some embodiments, the one or more processors and the non-transitory machine-readable storage medium are located in the wearable electronic device.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, the emphasis being placed upon illustrating the principles of the disclosure.

FIG. 1 illustrates an example of system environment for determining sleep stages based on change points according to embodiments.

FIG. 2 is a flow chart illustrating an example of a method of determining sleep stage using change points according to embodiments.

FIG. 3 is a flow chart illustrating an example of operating the sleep stage classifier on the change point events for each measure according to embodiments.

FIG. 4 is a flow chart illustrating an example of training the sleep stage classifier for each measurement according to embodiments.

FIG. 5A shows an example of a conversion of NN interval, tilt, and actigraphy time series into the change point events according to embodiments; and FIG. 5B shows an enlarged view of the change point events for each measure from FIG. 5A.

FIG. 6 shows an example of decoding the sleep stage from the change point events according to embodiments.

FIG. 7 is a simplified block diagram of an example of a computing system for implementing certain embodiments disclosed herein.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The disclosed embodiments relate to techniques for accurately detecting sleep stages of a subject (e.g., a human subject, a patient, an animal, (e.g., equine, canine, porcine, bovine, etc.), etc.) using timestamps of change events determined from the sensor data. The technique uses temporal information in the changes and the coupling between multiple sources to optimize classification. Various embodiments are described herein, including systems, methods, devices, modules, models, algorithms, networks, structures, processes, computer-program products, and the like.

As used herein, a sleep stage may refer to one or more phases or states of sleep. Each phase or state of sleep may refer to a phase or state having particular physiological characteristics. For example, potential sleep stages may include states such as wake and sleep. In a further example, potential sleep stages may also include different states of asleep, such as NI, N2, N3, N4, REM, and non-REM (NREM). In some examples, a potential sleep stage may correspond to multiple recognized phases or states of sleep. For example, NI, N2, N3, N4, REM, and non-REM (NREM) may comprise a single state, sleep stage.

In some examples, the determined stages of sleep may be further analyzed to determine sleep habits, sleep disorders (e.g., apnea, insomnia), sleep efficiency, sleep quality, among others, or a combination thereof. For example the determined sleep stages may be labeled qualitatively (e.g., descriptive phrase, such as “deep sleep,” “light sleep,” among others), quantitatively (e.g., score), or a combination thereof. In some embodiments, the disclosed embodiments may determine a disorder (e.g., insomnia) based on the determined sleep stage(s) for the period of time (e.g., a sleep session).

In some aspects, the disclosed embodiments may obtain, measure, detect, or receive one or more sets of sensor data (e.g., signals), such as photoplethysmographic (PPG) signal(s) and movement signal(s) from the respective sensor(s) (e.g., PPG sensor and accelerometer), included in a device worn on the individual (later referred to as “wearable device”). The disclosed embodiments may determine one or more sets of physiological measures, movement measures, among others, or a combination thereof from the one or more sets of sensor data. For example, the one or more physiological measures and/or movement measures may be any type of data derived from the measured signals. For example, the one or more physiological measures may include but is not limited to heart rate (e.g., Normal-to-Normal (NN) interval time series, heart rate variability, etc.), respiration rate, among others or a combination thereof. By way of another example, the one or more movement measures may include but is not limited to tilt angle, actigraphy, among others, or a combination thereof.

In some embodiments, the disclosed embodiments may determine one or more change point events (e.g., referred to as “change events”) for each set of measures. The one or more change point events may refer to one or more data points included in the measures indicating a change between sleep stages. Each change point may be associated with a respective time stamp. In some aspects, the disclosed embodiments may operate on the set of one or more change point events for each measure using a trained sleep stage classifier to determine a sleep stage associated with the individual. In some aspects, the sleep stage classifier may include a set of functions defining a likelihood that the individual is in a particular sleep stage, such as a sleep stage selected from a set of sleep stages.

Using the embodiments described herein, the classification models may be built, trained, and use change events for each measure to determine a sleep stage for a subject with high accuracy. The classification models may account for both excitatory and inhibitory influences from different domains. By using only change point events (e.g., event timestamps), the disclosed embodiments can require low-memory. Additionally, the disclosed embodiments may use little processing power and small memory space for storing the data. Thus, the disclosed embodiments can provide an immense memory savings for applications, and be implemented locally on devices with low processing power and small memory space (e.g., wearable electronic devices).

While some examples of the disclosure may be specific to sensors, such as PPG and accelerator sensors and measures, heart rate (e.g., NN interval series), tilt angle, and actigraphy, it will be understood that these examples are nonlimiting and that the methods and systems may be used to with other types of sensors data and/or measures, including but not limited to ECG, respiratory rate, among others, or a combination thereof.

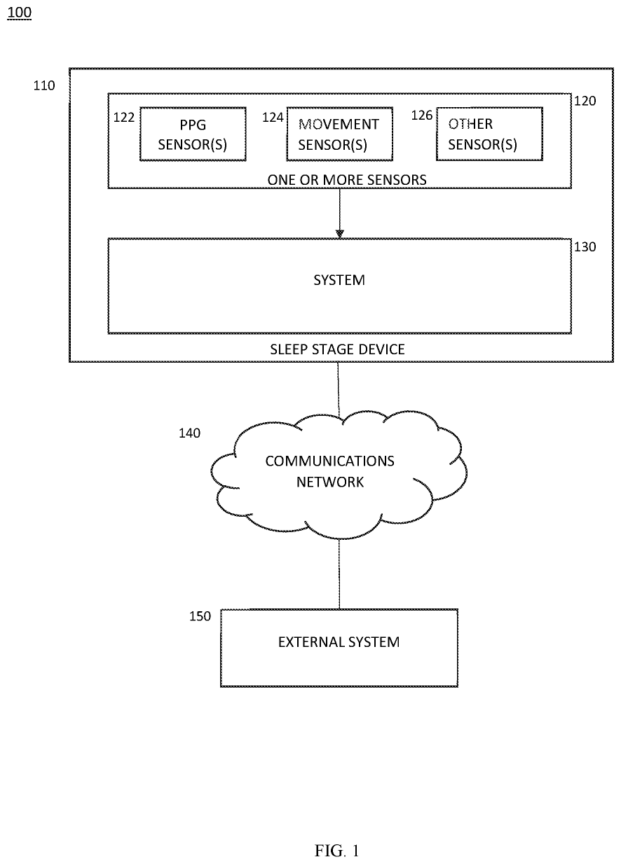

FIG. 1 depicts an example system environment 100 for determining one or more sleep stages using change events according to embodiments. In some embodiments, the sleep stage device 100 may include one or more sleep stage devices (e.g., sleep stage device 110) which may be associated with one or more individuals (e.g., user or subject). In some embodiments, the sleep stage device 100 may include one or more computing systems 130 for implementing processes consistent with the disclosed embodiments. The one or more computing systems 130 may be communicatively connected to one or more sensors 120. The one or more sensors 120 may be included within the sleep stage device 110 (as depicted in FIG. 1) or may be external to the sleep stage device 110. In some embodiments, the environment 100 may include one or more external computing devices/systems (e.g., external system 150). One or more communication networks (e.g., communication network 140) may communicatively connect one or more components of the environment 100.

In some embodiments, the sleep stage device 110 may include any computing or data processing device consistent with the disclosed embodiments. In some aspects, for example, the sleep stage device 110 may include a wearable device implemented with hardware components, sensors, and/or software applications running thereon for implementing the disclosed embodiments. In some embodiments, the sleep stage device 110 may incorporate the functionalities associated with a personal computer, a laptop computer, a tablet computer, a notebook computer, a hand-held computer, a personal digital assistant, a portable navigation device, a mobile phone, an embedded device, a smartphone, environmental sensor, and/or any additional or alternate computing device/system. The sleep stage device 110 may transmit and receive data across a communications network (e.g., the network 140).

By way of example, the communication network 140 can include one or more networks such as a data network, a wireless network, a telephony network, or any combination thereof. The data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, NFC/RFID, RF memory tags, touch-distance radios, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (WiFi), wireless LAN (WLAN), Bluetooth®, Internet Protocol (IP) data casting, satellite, mobile ad-hoc network (MANET), and the like, or any combination thereof. The sleep stage device 110 may further implement aspects of the disclosed embodiments without accessing other devices or networks, such as network 140 or the external device 150.

In some embodiments, the sleep stage device 110 may be associated with one or more individuals, such as user or a subject. In one example, a user/subject may wear the sleep stage device 110 (e.g., around the user's wrist, leg, chest, etc.) to perform one or more processes consistent with the disclosed embodiments, such as that described with reference to FIGS. 1-7. For example, a user/subject may use the sleep stage device 110 to input information, receive information, display information, and transmit information to and from other components in system environment 100, such as the external system 150. This information may include any data consistent with the disclosed embodiments.

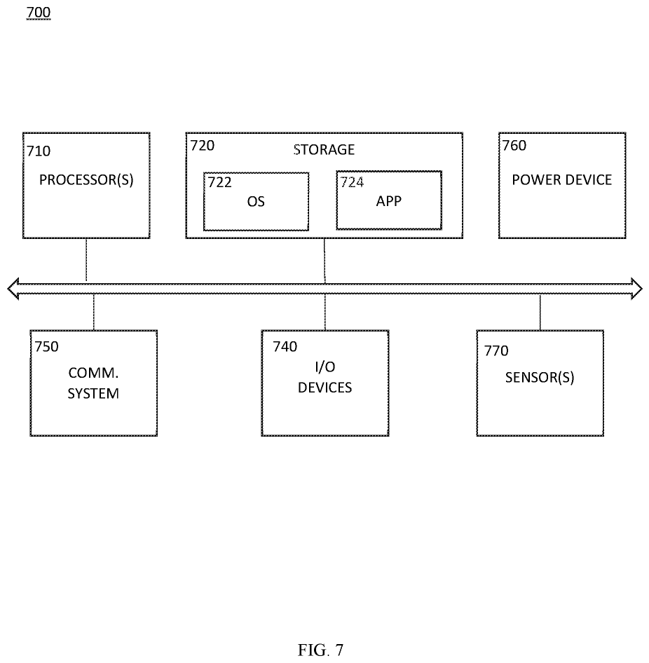

The sleep stage device 110 may include one or more computing systems 130 for processing, storing, receiving, obtaining, and/or transmitting information, such as computing system 700 described in connection with FIG. 7. In some aspects, the system 130 may be implemented with hardware components and/or software instructions to perform one or more operations consistent with the disclosed embodiments (e.g., the example embodiments described with reference to FIGS. 1-7). The software instructions may be incorporated into a single computer or any additional or alternative computing device/system (e.g., a single server, multiple devices etc.). The system 130 may also include or associate with distributed computing devices and computing systems, and may execute software instructions on separate computing systems by remotely communicating over a network (e.g., the communications network 140). The system 130 may also implement aspects of the disclosed embodiments without accessing other devices or networks, such as communications network 140. The sleep stage device 110 and/or the system 130 may also be implemented with one or more data storages for storing information consistent with the embodiments described below.

In some embodiments, the sleep stage device 110 may be configured to determine sleep stage(s) for the period of time using at least the change events determined from physiological and/or movement measures derived from the sensor data collected by the sensors 120. In some embodiments, the one or more sensors 120 may include a photoplethysmography (PPG) sensor 122, one or more movement sensors 124, one or more other sensors 126, or a combination thereof.

In some embodiments, the one or more sensors 120 may be implemented as hardware components within the sleep stage device 110, may reside external to the sleep stage device 110, or a combination thereof. For example, the one or more sensors 122, 124, and 126 and the computing system 130 may be housed in the same wearable electronic device or distributed between wearable electronic devices in different wearable electronic devices and/or one or more other electronic devices (e.g., mobile device, the external system 150, etc.) that may have connectivity to the sleep stage device 110 via the communication network 140.

In some embodiments, the wearable electronic device (also referred to as “wearable device”) may be a device that can be removably attached to a user. The wearable device may be implemented with hardware components (e.g., the computing system 130), one or more sensors (e.g., the sensors 120), and/or software applications running thereon for implementing the disclosed embodiments. In some embodiments, the wearable electronic device is worn on a body part, e.g., an arm, a wrist, an ankle, or a chest, etc., of the user, or embedded in a garment worn by the user. By way of example, the wearable electronic devices may include but is not limited to a smart watch, glasses, a headband, helmet, a smart phone attached using an attachment device (e.g., arm band), among others, or a combination thereof. Examples of the one or more other electronic devices include mobile phone, a cellular phone, a smart phone, a personal computer (PC), a server including hardware and software, a tablet, a smartphone, a desktop, a computer, a netbook, a laptop computer, a smart television, among others, or a combination thereof. FIG. 7 shows an example of a wearable electronic device/electronic device.

In some embodiments, the one or more sensors 120 may be disposed on a different device that communicates with the other sensors and/or the device 110. By way of example, that device may include, for example, a patch (e.g., adhesive patch, sticker, etc.)

In some embodiments, the one or more movement sensors 124 may include but are not limited to an accelerometer, gyroscope, among others, or a combination thereof. By way of example, the accelerometer may be configured to detect accelerations of body parts of the subject and be configured to detect motion (e.g., posture changes) of the subject by determining changes in average orientation of the accelerometer with respect to gravity.

In some embodiments, the one or more sensors 120 may also include one or more other sensors 126. In some embodiments, the one or more other sensors 126 may include but are not limited to a thermometer, location (such as GPS), galvanic skin response/electrodermal activity sensors, ECG sensor(s), electromyographic sensor(s), electroencephalographic sensor(s), phonocardiographic (PCG) sensor(s), acoustic sensor(s), optical sensor(s), ballistocaridographic sensor(s), video or camera sensor(s), off-body sensor(s) (e.g., radar sensor(s), video or camera sensors (s)), other sensors configured to collect biometric information, among others, or a combination thereof. By way of example, the electrocardiograph (ECG) sensors may include direct contact electrodes on the skin or capacitive contact; the opto-electrical photoplethysmography (PPG) measurements may include light source, e.g., a light emitting diode (LED) and photodetector (e.g. transistor/diode or a photodiode (PD)) as a receiver against the skin, LED and Photo diode arrays as transmitter-receiver pairs against the skin, a camera as a detector; the PCG sensors may include a Giant-Magneto-Resistance (GMR) sensors; the acoustic sensors may include an acoustic sensor based microphone; and the off-body sensors may include off-body devices such as radar, cameras, LIDAR, etc.

In some embodiments, the sleep stage device 110 may process the sensor data to determine one or more physiological and/or movement measures for a period of time. Using these measures, the sleep stage device 110 may convert the signals for each measure into a plurality of change point events (series) for each measure. Each change point may be associated with a timestamp. The sleep stage device 110 may use the change point event series to classify the sleep stage(s) for the period of time. By using only the change point events for each measure rather than the entire signals for each measure, the device 110 may utilize low processing power and small memory space to determine sleep stage(s) for a period of time.

Although the systems/devices of the environment 100 are shown as being directly connected, the sleep stage device 110 may be indirectly connected to one or more of the other systems/devices of the environment 100. In some embodiments, the device 110 may be only directly connected to one or more of the other systems/devices of the environment 100.

It is also to be understood that the environment 100 may omit any of the devices illustrated and/or may include additional systems and/or devices not shown. It is also to be understood that more than one device and/or system may be part of the environment 100 although one of each device and/or system is illustrated in the environment 100. It is further to be understood that each of the plurality of devices and/or systems may be different or may be the same. For example, one or more of the devices of the devices may be hosted at any of the other devices.

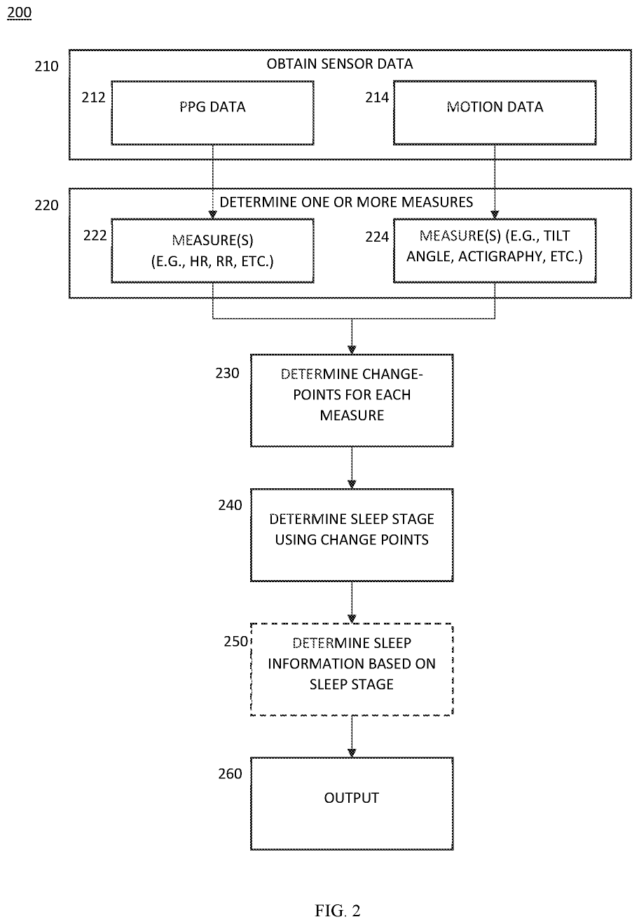

FIG. 2 shows a flow chart 200 illustrating an example of a method of detecting sleep stages using the change events according to certain embodiments. Operations described in flow chart 200 may be performed by a computing system, such as the system 130 of the device 110 described above with respect to FIG. 1 or a computing system described below with respect to FIG. 7. Although the flow chart 200 may describe the operations as a sequential process, in various embodiments, some of the operations may be performed in parallel or concurrently. In addition, the order of the operations may be rearranged. An operation may have additional steps not shown in the figure. In some embodiments, some operations may be optional. Embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

Operations in flow chart 200 may begin at block 210, the sleep stage device 110 may receive at least one set signals/data from the one or more sensors 120. For example, the set of signals may include a set of PPG signals 212 measured with the PPG sensor 122 and a set of accelerometer signal 214 measured with the accelerometer 124 of the device 110. In some embodiments, the set of signals may include one or more sets of additional signals from the one or more sensors 126, such as a GNSS sensor, a GPS receiver, a thermometer, an air pressure sensor, a blood pressure sensor, or any other sensor contemplated by the disclosed embodiments (e.g., the types of sensors described with connection with FIG. 1). As part of step 210, the sleep stage device 110 may receive these signals directly from the set of sensors 122 and 124 (e.g., as implemented within sleep stage device 110) and/or detect, measure, or otherwise derive them to form the set of signals described herein.

In some embodiments, at block 220, the sleep stage device 110 may determine one or more measures for each set of signals, for example, using any known techniques. For example, for the PPG data, at block 222, the sleep stage device 110 may determine a heart rate. The heart rate may include but is not limited to beat intervals, such as Normal-to-normal (NN) interval series. In some embodiments, a beat interval may reflect a duration of time between successive heartbeats reflected in the PPG signal. In some embodiments, additional and/or alternative measurements may be determined from the PPG signal including but not limited to respiratory rate (RR), heart rate variability, among others, or any combination thereof.

In some embodiments, the 3-axis accelerometer data may be converted to activity counts so as to determine actigraphy and tilt angle (e.g., the angle the watch was tilted form the flat position).

In some aspects, additional measures from the sensor data may be determined. For example, the respiration rate may be determined from the PPG data. In some embodiments, at block 230, the sleep stage device 110 may convert the three measurements (e.g., actigraphy, tilt angle, and heart rate) into change point events for every timestamp for each measurement. For example, the change point detection may detect changes in the mean and standard deviation, for example, using binary segmentation. For example, each measurement may be processed at one or more successive time points (e.g., every millisecond, two milliseconds, or other time points) to determine whether there is a point of change in the measurement with respect to the reference measurement above a threshold. That first point of change and subject point of change may be considered a series of change point events (also referred to as “change events” or “event streams”) for a measurement. It is noted that in some embodiments or aspects, processing of some time points can be omitted (e.g., every other time point or two of three time points). In some embodiments, different methods may be used to determine the change point events. For example, the change point events may be determined using Bayesian Online Changepoint Detection, Pruned Exact Linear Time, among others, or a combination thereof.

In some embodiments, the device 110 may process each measure to determine its respective change point events (also referred to as “change point event series”). For example, after processing the raw data to the change point events, the device 110 may no longer need to store the entire raw signal and may store only the change events for each measure for that time interval for that period; thereby reducing the memory needed to perform the techniques according to embodiments.

In this example, the change event series may be determined for each of actigraphy, tilt angle, and heart rate. In other examples, the change event series may be determined for alternative and/or additional measurements. By way of example, the change event series may be determined for respiratory rate/signal, another heart rate measurement (e.g., heart rate variability), among others, or any combination thereof.

At block 240, the sleep stage processing device 110 may including operating on the change events for each measurement using a sleep stage classifier to determine the one or more sleep stages associated with the period of time. In some embodiments, the sleep stage classifier may reflect a set of functions, parameters, computational weights, coefficients, etc., defining a likelihood that an individual is in a particular sleep stage (e.g., wake or sleep) based on a set of inputs, such as the change events for each measurement.

In some embodiments, the steep stage classifier may include a set of plurality of parameters (also referred to as “coefficients”) that is stored for each measure. In some embodiments, the set of plurality of parameters may include a sleep stage change event parameter (e.g., also referred to as “change event parameter”) (k), a history parameter (h), and one or more coupling parameters (c). The coupling parameters may relate to learned interactions between two measures (e.g., the respective measure and another measure). The change event parameter (also referred to as “sleep/wake stimulus” or “stimulus”) may act as a stimulus parameter representing the change between sleep stages associated with a specific measurement.

For example, if the measures include heart rate, actigraphy, and tilt angle, the classifier may include three sets of parameters that are individually applied to each measure. By way of example, for the change series for the heart rate measure, the parameters may include a sleep stage change event parameter, a history parameter, a first coupling parameter representing a relationship between the heart rate measure and the actigraphy measure, and a second coupling parameter representing a relationship between the heart rate measure and the tilt angle measure; for the change series for the actigraphy measure, the parameters may include a sleep stage change event parameter, a history parameter, a first coupling parameter representing a relationship between the actigraphy measure and the heart rate measure, and a second coupling parameter representing a relationship between the actigraphy measure and the tilt angle measure; and for the change series for the tilt angle measure, the parameters may include a sleep stage change event parameter, a history parameter, a first coupling parameter representing a relationship between the tilt angle measure and the actigraphy measure, and a second coupling parameter representing a relationship between the tilt angle measure and the heart rate measure. Each set of parameters may be specific to the measure. For example, the first coupling parameter for the heart rate measure may be different from the first coupling parameter for the actigraphy measure.

In some embodiments, the parameters for the sleep stage classifier may be learned from a training data set. A set of training data may be used to determine the corresponding parameters for each measure to estimate or predict the sleep stage of a person.

By example, the training data may be patient data that includes multi-channel sleep data for patients collected in a sleep center and corresponding data for these patients collected using the sensor(s) 120. The clinical database may include data from individuals with a variety of sleep conditions, such as insomnia, nocturnal frontal lobe epilepsy, REM behavior disorder, bruxism, and sleep apnea. In some embodiments, the training data may include sleep data collected in a sleep center for the subject/user of the device 110 resulting in a personalized trained model for the user.

For example, the sleep stage classifier may be a trained encoded model. For the sleep stage classifier, change events for each may be used to train the classifier, for example, using an encoding model. In some embodiments, the encoding model may include a plurality of filters for each measure. For example, for each measure, the encoding model may include a history filter, a sleep stage transition (e.g., sleep/wake stimulus) filter, and one or more coupling filters.

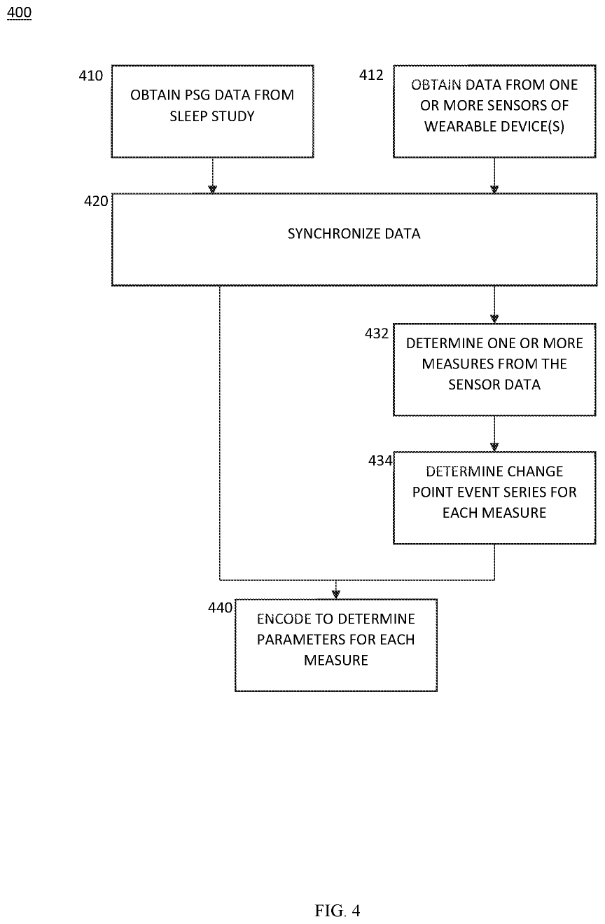

During the encoding, optimal filters may be selected using the training data. In some embodiments, the parameters may be determined by fitting a generalized linear model, such as Poisson Generalized Linear Model (GLM), to the training data. FIG. 4 shows a flow chart 400 illustrating an example of a method of generating a sleep stage classifier from the training data, using GLM, according to embodiments. In some embodiments, the sleep stage classifier may be trained using additional and/or alternative techniques.

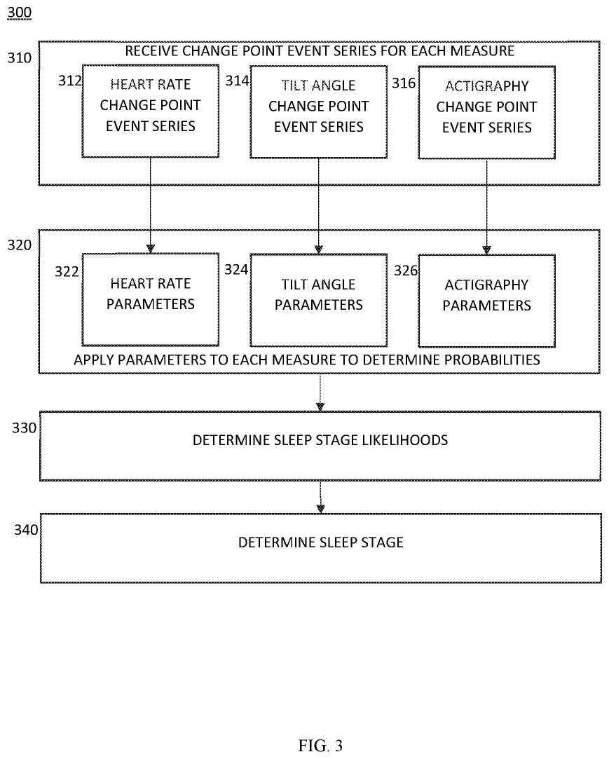

In some embodiments, the processing at block 240 may include processing each measure by applying the parameters to determine sleep stages for the period of time. In some embodiments, the processing at block 240 may include decoding the sleep stage from the change event series using a maximum likelihood estimation. FIG. 3 shows a flow chart illustrating an example of a method of operating a sleep stage classifier using maximum likelihood estimation. In some embodiments, the processing at block 240 may involve other known machine learning, such as spike neural network, other regression models, among others, or a combination thereof.

FIG. 3 is a flow chart 300 illustrating an example of a method of determining a sleep stage using the sleep stage classifier according to embodiments. Operations described in flow chart 300 may be performed by a computing system, such as the system 130 of the device 110 described above with respect to FIG. 1 or a computing system described below with respect to FIG. 7. Although flow chart 300 may describe the operations as a sequential process, in various embodiments, some of the operations may be performed in parallel or concurrently. In addition, the order of the operations may be rearranged. An operation may have additional steps not shown in the figure. In some embodiments, some operations may be optional. Embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

Operations in flow chart 300 may begin at block 310, the device 110 may receive the change point series for each measure for a period of time, for example, from block 230 or from storage. In this example, the measures may include change point events (series) for each measure (e.g., change points for heart rate 312, change point for tilt angle 314, and change points for actigraphy 316).

Next, at block 320, the set of parameters for each respective measure may be applied to the determined change events (from block 230) to determine a probability of a (observing) change event at that determined change event. For example, the set of parameters for heart rate 322 may be applied to change event series for heart rate 312, the set of parameters for tilt angle 324 may be applied to change event series for tilt angle 314, and the set of parameters for actigraphy 326 may be applied to actigraphy 316. This can result in a probability for each determined change event of the heart rate, a probability for each determined change event of the tilt angle and a probability for each determined change event of the actigraphy.

In some embodiments, each measure may have equal weight. After the probability is determined for each change event of each measure, the probabilities may be summed to determine the probabilities for each epoch or interval of the period of time. For example, each epoch may be 30 seconds. In other embodiments, the measures may have different weights.

Next, at block 330, the sleep stage likelihood estimation may be determined using the combined (or summed) probabilities for the period of time. In some embodiments, a maximum likelihood estimation may be applied to the probabilities for the period of time to determine an sleep stage likelihood for each epoch. The sleep stage likelihood may be a value representing a likelihood that the event is in one of the sleep stage(s).

Next, at block 340, the sleep stage for each timestamp (interval/epoch) of the period of time may be determined. In some embodiments, the processing at block 340 may include comparing the sleep stage likelihood for each epoch to one or more stored (stage likelihood) thresholds determine the state associated with that epoch. For example, if the sleep stages include a first stage (wake) and a second stage (sleep), if the likelihood for an epoch is above the threshold, that epoch may be classified as sleep (or one of the stages associated with sleep) and if the likelihood for a epoch is below the threshold, the epoch may be classified as wake. The interval/epoch may include but is not limited to 10 seconds, 20 seconds, 30 seconds, among others, or any combination thereof.

Next, after thresholding, the processing at block 340 may include converting the likelihood values for each epoch into the associated sleep stage for that epoch. This may result in the sleep stage(s) over the period of time.

Referring back to FIG. 2, the processing at block 240 may determine one or more periods of one or more sleep stages for the sleep session using the determined sleep stages for each epoch. For example, after applying the classifier (e.g., FIG. 3), the processing at step 240 may determine that a subject had a certain number of minutes of movement (wake), a certain number of minutes of sleep during the period of time.

After determining the associated sleep stage(s) over the period of time, the determined sleep stages may optionally be further processed at block 250 to determine qualitative and/or quantitative sleep information. For example, at block 250, the quantitative sleep information may include one or more scores or metrics (e.g., overall restlessness, total sleep time metric, unified sleep score, long wakes metric, heart rate metric, deep sleep metric, breathing disturbances metric, among others, or a combination thereof). The qualitative sleep information may categorize the sleep as light sleep, deep sleep, etc. based on the number of periods of sleep and the number of periods of wake during a sleep session.

In some embodiments, the processing at block 250 may include determining one or more disorders based on the number of periods of sleep and the number of periods of wake during a sleep session. For example, the insomnia may be determined used these periods.

Next, at block 260, the device 110 may output the determined sleep stage and/or sleep information associated with the period of time (e.g., sleep session). For example, the device 110 may store the determined sleep stage and/or sleep information associated with a sleep session. In some embodiments, the device 110 may output the determined sleep stage and/or associated sleep information. For example, the output may include generating a graphical representation of the sleep information and/or stages to be displayed on the device 110 or another coupled electronic device (e.g., mobile smart device).

FIG. 4 is a flow chart illustrating an example 400 of a method of generating a sleep stage classifier from the training data according to embodiments. Operations described in flow chart 400 may be performed by a computing system, such as the system 130 of the device 110 described above with respect to FIG. 1 or a computing system described below with respect to FIG. 7. Although flow chart 400 may describe the operations as a sequential process, in various embodiments, some of the operations may be performed in parallel or concurrently. In addition, the order of the operations may be rearranged. An operation may have additional steps not shown in the figure. In some embodiments, some operations may be optional. Embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

Operations in flow chart 400 may begin at blocks 410 and 420, a computing device/system may receive multi-channel sleep data for patients collected in a sleep center and at the corresponding data for these patients collected, for example, using the sensors 120, respectively. For example, the sensor data obtained at block 420 may include accelerometer and PPG data.

Next, at block 430, the computing device may synchronize the sleep data from block 410 and the sensor data from block 420. After, the computing device may determine or more measures from the sensor data 432, for example, as discussed above with respect to block 210. For example, heart rate may be determined from the PPG data, and tilt angle and actigraphy may be determined from the accelerometer data.

Next, at block 434, change point event series may be determined for each measure, for example, as discussed above with respect to block 230.

Next, at block 440, the change point event series from block 434 and the synchronized sleep data from step 420 may be encoded, to determine a set of parameters for each measure. During the encoding, optimal filters may be selected using the training data. For example, the filters may include a history filter, one or more coupling filters, and a sleep change event filter to determine the history parameter, the one or more coupling parameters, and the change event parameter, respectively, for each measure. For example, the processing generating the event streams at block 230 can be viewed as a Poisson Generalized Linear Model (GLM) and the parameters (filter coefficients) may be determined by fitting the GLM to the training data.

After the parameters (coefficients) for each measure are determined, the parameters may be stored locally on the device 110, for example, for use in the methods described in FIGS. 2 and 3

### EXAMPLES

As described above, techniques (referred to in this example a “Change Point Decoder” (CPD)) disclosed herein can be used to detect one or more sleep stages of subject for a period of time using a sleep stage classifier based on change events for one or more measures derived from movement and PPG data collected by an accelerometer and PPG sensor, respectively, of a wearable electronic device. An experiment has been conducted using techniques disclosed herein to determine a trained classifier to determine one or more sleep stages for a period of time. Its performance to the well-established Oakley algorithm (OA) relative to polysomnography (PSG) in elderly men with disordered sleep.

Overnight in-lab polysomnography, PPG, and accelerometer data were collected from 102 male participants simultaneously. (mean age=68.56, SD=1.93). Participants underwent a polysomnography (PSG) study and wore wearable device (Empatica E4, Empatica; Cambridge, Mass.) simultaneously during the overnight recording. Empatica watch acquired PPG and 3-axes accelerometer signals with sampling rates 64 Hz and 32 Hz respectively.

The study population was assigned to four groups according to their Apnea-Hypopnea Index (AHI) and Periodic Limb Movement Index (PLMI) as follows:

Group 1: Subjects with AHI<15 and PLMI<15

Group 2: Subjects with AHI≥15 and PLMI<15

Group 3: Subjects with AHI<15 and PLMI≥15

Group 4: Subjects with AHI≥15 and PLMI≥15

All the data were randomly split into two sets, with 70 subjects assigned to the training set and 32 subjects assigned to testing. Table 1 show ages and PSG-defined sleep efficiency in both sets. Two-sample Kolmogorov tests were performed for age, AHI, PLMI, and sleep efficiency of the subjects in the training and testing sets. Differences in these measures between the sets were not statistically significant, suggesting that the training set is representative of the testing set.

A series of preprocessing steps were applied to PPG and accelerometer signals to convert these signals into a sequence of events. Initially, the Empatica E4 timestamp was synchronized with the PSG timestamp. The next preprocessing step consisted of converting the PPG signal to Normal-to-Normal (NN) beat interval time series and three-axis accelerometer data to actigraphy and angle time series. PPG data were preprocessed using PhysioNet Cardiovascular Signal Toolbox. First, peak detection was performed using the qppg method provided with the toolbox, and the data was converted to peak-to-peak (PP) interval time series. Then, non-sinus intervals were detected and removed by measuring the change in the current PP interval from the previous PP interval and excluding intervals that change by more than 20%. PP intervals outside of physiologically possible range were also removed to obtain NN interval time series, which was filtered using a Kalman filter to reduce noise.

Raw three-axis accelerometer data were converted to activity counts following the approach described by Borazio et al. See, Borazio M, Berlin E, Kucukyildiz N, Scholl P, Van Laerhoven K. Towards benchmarked sleep detection with wrist-worn sensing units. In: IEEE; 2014:125-134. Activity counts are the output format of most commercial actigraphy devices; data are summarized over 30-second epochs or time intervals. This conversion compressed information, reduced required memory for storing data, and eliminated artifacts and noise in raw data. Z-axis actigraphy data were filtered using a 0.25-11 Hz passband to eliminate extremely slow or fast movements. The maximum values inside 1-second windows were summed for each 30-second epoch of data to obtain the activity count for each epoch.

Lastly, a tilt angle time series was derived from the raw accelerometer data to capture information that is not present in the activity count time series. Specifically, tilt angle, which is the angle between the gravitational vector measured by the accelerometer and the initial orientation with the gravitational field pointing downwards along the z-axis, can be calculated from the accelerometer reading as

$$ρ = \frac{{a}_{z}}{\sqrt{{a}_{χ}^{2} + {a}_{y}^{2} + {a}_{z}^{2}}} \qquad ( 1 )$$

where ρ is the tilt angle and aₓ, a_{y}, and a_{z} are the readings from x, y, and z axes of the accelerometer respectively.

After obtaining the NN interval, actigraphy and tilt angle time series, change point detection techniques were applied to detect significant changes. Binary Segmentation (BiS) was used on the preprocessed actigraphy, tilt angle, and NN interval time series to detect significant changes in the mean and standard deviation.

The procedure started by searching for a change point τ in the input signal S={x₁, x₂, . . . , x_{N}} that satisfied the condition

C_{S}_{_{1:τ}}+C_{S}_{_{τ+1:N+β<C}}_{S}_{_{1:N}} (2)

where C is a cost function and β is a penalty term that reduces overfitting. If the condition in Eq. 2 is met, τ becomes the first estimated change point, and S_{1:τ} and S_{τ+1:N} become the first subsequences. The process continued within these subsequences until data cannot be divided any further. Cost function in the above equation is given by

$${C}_{{S}_{{τ}_{i - {1}^{: {τ}_{i}}}}} = - 2 log ℒ ( θ │ {S}_{{τ}_{i - {1}^{: {τ}_{i}}}} ) \qquad ( 3 )$$

where [symbol] is the likelihood function.

In this study, changes more than 10° tilt angle were used as a change point in the tilt angle time series. In this way, all signals were represented as event sequences of the form t₁ᵢ, t_{2,i}, . . . , t_{n,i} where n∈Z+ was the index of the change point, i∈{1, 2, 3} was the type of time series change point occurred, and t∈R>0 denoted the time.

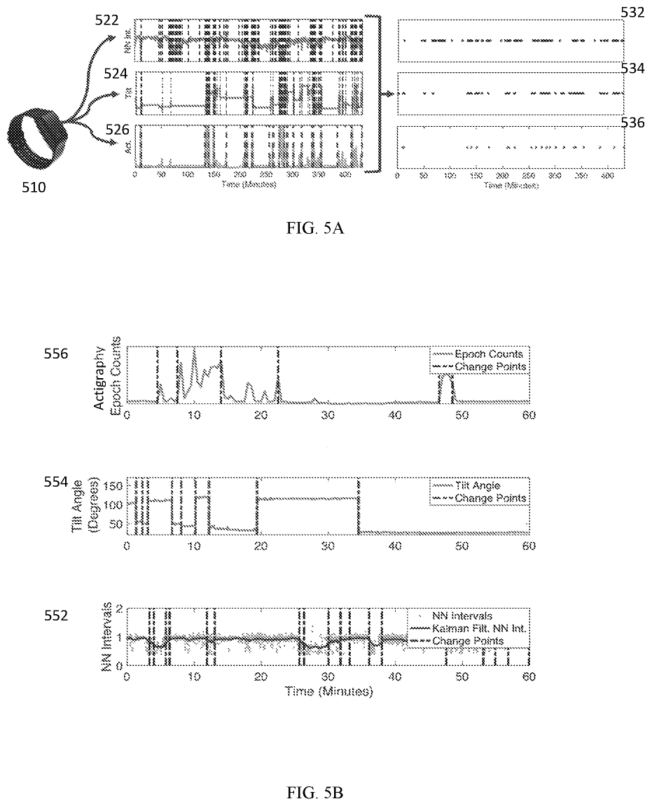

FIGS. 5A and 5B illustrates the conversion of Conversion of NN interval, tilt, and actigraphy time series into point processes. As shown in the FIG. 5A, using the PPG and accelerometer sensor data obtained from the wearable device 510 and the determined measures, the NN interval measures determined from the PPG data may be converted to change point series 522, the tilt angle measures determined from the accelerometer data may be converted to change point series 524, and the actigraphy measures determined from the accelerometer data may be converted to change point series 526.

FIG. 5A shows the signals and detected change events as dashed lines in change point streams (series) 522, 524, and 526. Arrival times of each detected change event t_{n,i} are shown as dots in the respective change event streams 532, 534, and 536. FIG. 5B shows an enlarged view of the detected change point series shown in FIG. 5A. In FIG. 5B, the change points are visualized as dash lines. The detected change point streams 552 for NN intervals corresponds to the detected change point streams 522; the detected change point streams 554 for the tilt angle corresponds to the detected change points 524; and the detected change point streams 556 for the actigraphy corresponds to the detected change point streams 526.

Next, the change events occurring in different signals were modeled in an encoding step. The sleep/wake signal through the night was thought as the stimulus driving the changes in the NN time series and actigraphy signals collected by the wearable device. The information in the change point time series was used to train the encoding model. The model included a history filter, coupling filters, and a stimulus filter. In the encoding step, the optimal filters were selected using the training data.

For example, the instantaneous firing rate of NN time series can be expressed as

r_{NN}(t)=f(k_{NN}·x(t)+h·z_{NN,history}(t)+c_{NN,act}·z_{act}(t)+c_{NN,angle}·z_{angle}(t)) (4)

where x(t) was the sleep/wake stimulus that drives the changes in the signals. k, h and c are stimulus, history, and coupling filters respectively. z_{NN,history} represented the history of the NN time series while z_{act} and z_{angle} were the windows of actigraphy and angle time series. f was selected as the exponential function and it converted the summation into probability of spiking. This set of four filters were fitted for each actigraphy, angle, and NN time series. Filter coefficients were calculated by using “glmfit” function from MATLAB.

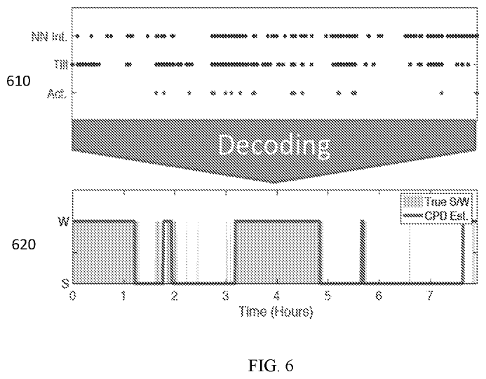

The process generating the event streams was viewed as a Poisson Generalized Linear Model (GLM) and filter coefficients were estimated by fitting GLM to the data. This generalized linear model approach allowed for both excitatory and inhibitory interactions between signals. Coupling filters facilitated modeling known interactions between heart rate and movement signals. The encoding process was repeated for NN interval, actigraphy, and tilt angle time series. Next, in a decoding step, sleep/wake states x(t) was decoded back from the patterns of change observed in the signals, as shown in FIG. 6. The decoding used the trained model from the encoding step and tries to estimate if the subject is asleep or wake, given the changes in the input signals. The log-likelihood function for events from a multidimensional process was given by

[symbol]

(t_{1,i},t_{2,i}, . . . ,t_{n,i})=log p(z|x,θ)==Σ_{i,n} log λᵢ(t_{n,i})−Σᵢ∫₀^{t}λᵢ(t)dt (5)

where θ={k, h, c} represented model parameters from encoding step, x was the sleep/wake stimulus, z was the event streams. The posterior probability of the sleep/wake stimulus given the event streams was

log p(x|z)=log p(z|x)+log p(x) (6)

Then, using Eq. 5 and Eq. 6, penalized maximum likelihood estimate of the sleep/wake stimulus was calculated by minimizing

$${x}_{e s t} = {arg min}_{x} ( - log p ( x │ z ) + λ {‖ x ‖}_{T V} ) \qquad ( 7 )$$

where zₑₛₜ is the estimate sleep/wake and z is the change point time series and log p (x|z) is the log-probability of sleep/wake states given the observed change events. The likelihood was regularized with the Total Variation (TV) norm to prevent overfitting and preserve step-like properties of the sleep/wake stimulus. After estimation, the output xₑₛₜ is thresholded and converted back to binary sleep/wake detection.

FIG. 6 shows an example of a plot showing encoding sleep/wake states from the event streams. Top plot 610 shows event streams from NN intervals, tilt angle, and actigraphy. Bottom plot 620 shows the true sleep/wake states and the (determined) estimate.

Five-fold cross-validation was used to tune hyperparameters and validate the model. Regularization parameter (L) and threshold resulting in the highest F1 score were selected using four folds of data as training, and the performance was reported on the remaining part of data. F1 score was used as the model selection metric because it takes into account both precision and recall. In sleep/wake detection task, precision indicates how many epochs in the detected wake are correct. Recall refers to the percentage of total wake epochs results correctly classified. Therefore F1 score, which combines precision and recall, proved to be a useful metric for this imbalanced classification scenario where wake is the minority class.

The Oakley sleep/wake detection method was also implemented on the same dataset to allow a fair comparison with the proposed technique. Actigraphy data were weighted and summed as follows

Aᵢ=0.04E₍ᵢ₋₄₎+0.04E₍₁₋₃₎+0.2E₍ᵢ₋₂₎+0.2E₍ᵢ₋₁₎+2E₍ᵢ₎+0.2E₍ᵢ₊₁₎+0.2E₍ᵢ₊₂₎+0.04E₍ᵢ₊₃₎+0.04E₍ᵢ₊₄₎ (8)

where i denotes the current epoch index and E denotes the actigraphy count in the epoch. Then Aᵢ is compared to a predefined threshold to identify sleep/wake. In commercially available Actiwatch devices, there are three different thresholds: low (20), medium (40), and large (80). Since the wearable device is different in this study, it could result in an actigraphy time series with a different amplitude range than Actiwatch and thresholds may not apply. Therefore, the threshold was selected using the training data to maximize F1 score. Results of both optimized threshold and medium setting are reported for comparison.

To evaluate the performance of the model, standard metrics such as sleep accuracy, wake accuracy, and total accuracy were calculated. F1 score was used both for hyper-parameter selection as described above and for evaluating the algorithms. Also, the regularization parameter of CPD was fixed to the value selected using the F1 score and sweep thresholds for both methods to derive ROC and Precision-Recall curves. Cohen's Kappa was also calculated to measure inter-rater reliability between PSG study and the algorithms. Furthermore, sleep-wake statistics including Wake After Sleep Onset (WASO), Sleep Onset Latency (SOL), Sleep Efficiency, and the number of sleep wake transitions were calculated. WASO was defined as the minutes awake during the sleep period after sleep onset (defined as the first 30-second epoch of any stage of sleep). Sleep Onset Latency was calculated as the time from lights out until sleep onset in minutes. Sleep efficiency was defined as the percent of time scored as sleep during the sleep period subsequent to sleep onset. For training set performance evaluation, models were trained and validated using leave-one-out cross validation within training set. For testing set performance evaluation, final model was trained using the subjects in the training set with selected hyperparameters and tested on the testing set. Using individual signal models without the coupling filters between different domains was also tested in the same manner in order to assess the contribution of each signal and the coupling filters to the performance.

Hyperparameters selected on training set for CPD are 1-minute window size, regularization parameter of 2, and threshold of 0.22. For the OA method, threshold optimized with F1 score on the training set is equal to 70. Concordance between PSG and the methods are evaluated on testing set. The mean across subjects for total accuracy, sleep accuracy, wake accuracy, Kappa, F1 score, WASO, and SE are shown in Table 1 for both methods. For WASO, SE and the number of sleep wake transitions, the error is calculated as the PSG gold standard minus estimated value.

As shown in Table 1, the CPD method achieved greater accuracy for wake accuracy, Kappa, and F1 Score for both training and test sets. The difference between wake accuracy was statistically significant (P<0.05) for the methods in both training and test sets. It can also be seen that OA overestimated WASO while wake accuracy is low. Note that the CPD method exhibited lower WASO error in the analyses.

TABLE 1
Sleep/wake identification performances in the Testing Set

| | Testing Set | | | | | | | |
|---|---|---|---|---|---|---|---|---|
| | OA | | | | CPD | | | |
| | Mean (SD) | | 95% CI | | Mean (SD) | | 95% CI | |
| Total Accuracy | 0.76 | (0.09) | [0.72, | 0.79] | 0.72 | (0.14) | [0.67, | 0.77] |
| Sleep Accuracy | 0.85 | (0.12) * | [0.80, | 0.89] | 0.70 | (0.19) | [0.63, | 0.76] |
| Wake Accuracy | 0.54 | (0.20) | [0.47, | 0.62] | 0.74 | (0.20) * | [0.66, | 0.81] |
| Kappa | 0.39 | (0.17) | [0.33, | 0.45] | 0.40 | (0.24) | [0.31, | 0.49] |
| F1 Score | 0.59 | (0.14) | [0.54, | 0.64] | 0.62 | (0.20) | [0.55, | 0.70] |
| WASO Error (min.) | −9.95 | (63.75) | [−32.94, | 13.03] | 7.66 | (67.34) | [−16.62, | 31.94] |
| SE Error (%) | −0.03 | (14.93) | [−5.42, | 5.35] | 2.09 | (16.81) | [−3.97, | 8.15] |
| SOL Error (min.) | 28.64 | (36.84) | [15.36, | 41.92] | −22.86 | (58.68) * | [−44.01, | −1.7] |

* Wilcoxon signed-rank comparison of two methods, 5% significance level.
Abbreviations: CI, Confidence Interval; SD, Standard Deviation.

The CPD approach used a combination of movement-related and physiological signals, making it possible to overcome some of the limitations of previous algorithms based solely on actigraphy. For instance, the results demonstrate that the CPD method does not overestimate sleep and has high wake detection performance. Therefore, the CPD method can provide an unbiased solution to sleep/wake detection. The CPD modeled time series of discrete change events derived from wearable device signals and outputted a score of wakefulness which can be used to investigate gradual transitions between sleep and wake states within the epochs.

A significant improvement in wake accuracy was observed by using the CPD. Higher wake accuracy also resulted in lower WASO error for both training and test sets with the CPD. The OA method overestimated WASO and had lower wake detection accuracy, even though the threshold parameter was optimized during training. This outcome indicated that the Oakley algorithm misclassified sleep epochs as wake while being unable to recognize true wake epochs. Accurate estimates of WASO could become especially important in monitoring populations with difficulties falling or staying asleep. For example, WASO duration has been used as a diagnostic criterion for insomnia.

The CPD solely used the timestamps of change events to predict sleep/wake. The size of the dataset (accelerometer and PPG signals from all participants) was 6.91 GB. If the signals in this dataset are stored as event streams, the required memory reduced to only 1.3 MB, 0.02% of the original dataset size. Therefore, the method according to embodiments could result in immense memory savings for applications with more data streams or in long-term studies.

CPD provides higher wake detection accuracy when compared to a solely actigraphy-based method. Techniques according to the disclosure can thus provide high wake detection accuracy, and this could enable investigating the vital role of awakenings during the night in various psychological disorders. The CPD method requires low-memory in the wearable devices and therefore can be beneficial in long-term studies. Moreover, the CPD can adapt to different and novel devices and signals.

FIG. 7 depicts a block diagram of an example computing system 700 for implementing certain embodiments. For example, in some aspects, the computer system 700 may include computing systems associated with a device (e.g., the computing system 130 of the device 110) performing one or more processes (e.g., FIGS. 2-4) disclosed herein. The block diagram illustrates some electronic components or subsystems of the computing system. The computing system 700 depicted in FIG. 7 is merely an example and is not intended to unduly limit the scope of inventive embodiments recited in the claims. One of ordinary skill in the art would recognize many possible variations, alternatives, and modifications. For example, in some implementations, the computing system 700 may have more or fewer subsystems than those shown in FIG. 7, may combine two or more subsystems, or may have a different configuration or arrangement of subsystems.

In the example shown in FIG. 7, the computing system 700 may include one or more processing units 710 and storage 720. The processing units 710 may be configured to execute instructions for performing various operations, and can include, for example, a micro-controller, a general-purpose processor, or a microprocessor suitable for implementation within a portable electronic device, such as a Raspberry Pi. The processing units 710 may be communicatively coupled with a plurality of components within the computing system 700. For example, the processing units 710 may communicate with other components across a bus. The bus may be any subsystem adapted to transfer data within the computing system 700. The bus may include a plurality of computer buses and additional circuitry to transfer data.

In some embodiments, the processing units 710 may be coupled to the storage 720. In some embodiments, the storage 720 may offer both short-term and long-term storage and may be divided into several units. The storage 720 may be volatile, such as static random access memory (SRAM) and/or dynamic random access memory (DRAM), and/or non-volatile, such as read-only memory (ROM), flash memory, and the like. Furthermore, the storage 720 may include removable storage devices, such as secure digital (SD) cards. The storage 720 may provide storage of computer readable instructions, data structures, program modules, audio recordings, image files, video recordings, and other data for the computing system 700. In some embodiments, the storage 720 may be distributed into different hardware modules. A set of instructions and/or code might be stored on the storage 720. The instructions might take the form of executable code that may be executable by the computing system 700, and/or might take the form of source and/or installable code, which, upon compilation and/or installation on the computing system 700 (e.g., using any of a variety of generally available compilers, installation programs, compression/decompression utilities, and the like), may take the form of executable code.

In some embodiments, the storage 720 may store a plurality of application modules 724, which may include any number of applications, such as applications for controlling input/output (I/O) devices 740 (e.g., a sensor (e.g., sensor(s) 770, other sensor(s), etc.)), a switch, a camera, a microphone or audio recorder, a speaker, a media player, a display device, etc.). The application modules 724 may include particular instructions to be executed by the processing units 710. In some embodiments, certain applications or parts of the application modules 724 may be executable by other hardware modules, such as a communication subsystem 750. In certain embodiments, the storage 720 may additionally include secure memory, which may include additional security controls to prevent copying or other unauthorized access to secure information.

In some embodiments, the storage 720 may include an operating system 722 loaded therein, such as an Android operating system or any other operating system suitable for mobile devices or portable devices. The operating system 722 may be operable to initiate the execution of the instructions provided by the application modules 724 and/or manage other hardware modules as well as interfaces with a communication subsystem 750 which may include one or more wireless or wired transceivers. The operating system 722 may be adapted to perform other operations across the components of the computing system 700 including threading, resource management, data storage control, and other similar functionality.

The communication subsystem 750 may include, for example, an infrared communication device, a wireless communication device and/or chipset (such as a Bluetooth® device, an IEEE 802.11 (Wi-Fi) device, a WiMax device, cellular communication facilities, and the like), NFC, ZigBee, and/or similar communication interfaces. The computing system 700 may include one or more antennas (not shown in FIG. 7) for wireless communication as part of the communication subsystem 750 or as a separate component coupled to any portion of the system.

Depending on desired functionality, the communication subsystem 750 may include separate transceivers to communicate with base transceiver stations and other wireless devices and access points, which may include communicating with different data networks and/or network types, such as wireless wide-area networks (WWANs), WLANs, or wireless personal area networks (WPANs). A WWAN may be, for example, a WiMax (IEEE 802.9) network. A WLAN may be, for example, an IEEE 802.11x network. A WPAN may be, for example, a Bluetooth network, an IEEE 802.15x, or some other types of network. The techniques described herein may also be used for any combination of WWAN, WLAN, and/or WPAN. In some embodiments, the communications subsystem 750 may include wired communication devices, such as Universal Serial Bus (USB) devices, Universal Asynchronous Receiver/Transmitter (UART) devices, Ethernet devices, and the like. The communications subsystem 750 may permit data to be exchanged with a network, other computing systems, and/or any other devices described herein. The communication subsystem 750 may include a means for transmitting or receiving data, such as identifiers of portable goal tracking devices, position data, a geographic map, a heat map, photos, or videos, using antennas and wireless links. The communication subsystem 750, the processing units 710, and the storage 720 may together comprise at least a part of one or more of a means for performing some functions disclosed herein.

The computing system 700 may include one or more I/O devices 740, such as sensors 770, a switch, a camera, a microphone or audio recorder, a communication port, or the like. For example, the I/O devices 740 may include one or more touch sensors or button sensors associated with the buttons. The touch sensors or button sensors may include, for example, a mechanical switch or a capacitive sensor that can sense the touching or pressing of a button.

In some embodiments, the I/O devices 740 may include a microphone or audio recorder that may be used to record an audio message. The microphone and audio recorder may include, for example, a condenser or capacitive microphone using silicon diaphragms, a piezoelectric acoustic sensor, or an electret microphone. In some embodiments, the microphone and audio recorder may be a voice-activated device. In some embodiments, the microphone and audio recorder may record an audio clip in a digital format, such as MP3, WAV, WMA, DSS, etc. The recorded audio files may be saved to the storage 720 or may be sent to the one or more network servers through the communication subsystem 750.

In some embodiments, the I/O devices 740 may include a location tracking device, such as a global positioning system (GPS) receiver. In some embodiments, the I/O devices 740 may include a wired communication port, such as a micro-USB, Lightning, or Thunderbolt transceiver.

The I/O devices 740 may also include, for example, a speaker, a media player, a display device, a communication port, or the like. For example, the I/O devices 740 may include a display device, such as an LED or LCD display and the corresponding driver circuit. The I/O devices 740 may include a text, audio, or video player that may display a text message, play an audio clip, or display a video clip.

The computing system 700 may include a power device 760, such as a rechargeable battery for providing electrical power to other circuits on the computing system 700. The rechargeable battery may include, for example, one or more alkaline batteries, lead-acid batteries, lithium-ion batteries, zinc-carbon batteries, and NiCd or NiMH batteries. The computing system 700 may also include a battery charger for charging the rechargeable battery. In some embodiments, the battery charger may include a wireless charging antenna that may support, for example, one of Qi, Power Matters Association (PMA), or Association for Wireless Power (A4WP) standard, and may operate at different frequencies. In some embodiments, the battery charger may include a hard-wired connector, such as, for example, a micro-USB or Lightning® connector, for charging the rechargeable battery using a hard-wired connection. The power device 760 may also include some power management integrated circuits, power regulators, power convertors, and the like.

In some embodiments, the computing system 700 may include one or more sensors 770. The sensors 770 may include, for example, the sensors 122, 124, and/or 126 as described above. For example, the sensors may include a PPG sensor and accelerometer.

The computing system 700 may be implemented in many different ways. In some embodiments, the different components of the computing system 700 described above may be integrated to a same printed circuit board. In some embodiments, the different components of the computing system 700 described above may be placed in different physical locations and interconnected by, for example, electrical wires. The computing system 700 may be implemented in various physical forms and may have various external appearances. The components of computing system 700 may be positioned based on the specific physical form.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

The foregoing method descriptions and the process flow diagrams are provided merely as illustrative examples and are not intended to require or imply that the operations of various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an” or “the” is not to be construed as limiting the element to the singular.

While the terms “first” and “second” are used herein to describe data transmission associated with a subscription and data receiving associated with a different subscription, such identifiers are merely for convenience and are not meant to limit various embodiments to a particular order, sequence, type of network or carrier.

Various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such embodiment decisions should not be interpreted as causing a departure from the scope of the claims.

The hardware used to implement various illustrative logics, logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing systems, (e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more example embodiments, the functions described may be implemented in hardware, software, firmware, or any combination thereof. If implemented in software, the functions may be stored as one or more instructions or code on a non-transitory computer readable medium or non-transitory processor-readable medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable medium and/or computer-readable medium, which may be incorporated into a computer program product.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms, “and” and “or” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AC, BC, AA, ABC, AAB, AABBCCC, and the like.

Further, while certain embodiments have been described using a particular combination of hardware and software, it should be recognized that other combinations of hardware and software are also possible. Certain embodiments may be implemented only in hardware, or only in software, or using combinations thereof. In one example, software may be implemented with a computer program product containing computer program code or instructions executable by one or more processors for performing any or all of the steps, operations, or processes described in this disclosure, where the computer program may be stored on a non-transitory computer readable medium. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques, including, but not limited to, conventional techniques for inter-process communications, and different pairs of processes may use different techniques, or the same pair of processes may use different techniques at different times.

The disclosures of each and every publication cited herein are hereby incorporated herein by reference in their entirety.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

1. A method for determining a sleep stage, comprising:
obtaining at least one set of sensor data generated by one or more sensors for a period of time;
generating at least two measures from the at least one set of sensor data;
determining a series of change point events for each measure for the period of time; and
determining a sleep stage for each interval of the period of time from at least two sleep stages by processing the series of change point events for each measure using a sleep stage classifier;
wherein the sleep stage classifier includes a set of parameters for each measure, the set of parameters for each measure including one or more coupling parameters, each coupling parameter being related to the cross-correlation between the each measure and another one of the measures.

2. The method according to claim 1, wherein the one or more sensors are of a wearable electronic device.

3. The method according to claim 2, wherein the one or more sensors includes a photoplethysmographic (PPG) sensor and an accelerometer.

4. The method according to claim 3, wherein:
the at least two measures include actigraphy, tilt angle, and heart rate;
the determining the at least two measures includes:
determining the heart rate from the sensor data from the PPG sensor; and
determining the actigraphy and the tilt angle from the sensor data from the accelerometer.

5. The method according to claim 1, wherein the one or more sleep stages includes a sleep stage and a wake stage.

6. The method according to claim 1, wherein the set of parameters for each measure includes a sleep stage change event parameter and a history parameter.

7. The method according to claim 3, wherein:
the measures includes three measures; and
the set of parameters for each measure includes two coupling parameters.

8. The method according to claim 1, wherein the determining one or more sleep stages for each interval of the period of time includes:
applying the set of parameters for each measure to respective series of change point events to determine a probability of a change event; and
determining a probability of a change event for each interval of the period of time using each probability for each measure.

9. The method according to claim 8, wherein the determining one or more sleep stages for each interval of the period of time includes:
determining a sleep stage likelihood for each interval using the probability of the change event for each interval of time of the period of time; and
determining the sleep stage for each interval of time of the period of time from the sleep stage likelihood.

10. The method according to claim 1, further comprising:
determining sleep information using the sleep stage for each interval of the period of time.

11. A system, comprising:
a wearable electronic device to be worn by a use, the wearable electronic device including one or more sensors configured to generate sensor data;
one or more processors; and
a non-transitory machine readable storage medium storing computer-executable instructions which, when executed by the one or more processors, cause the one or more processors to:
obtaining at least one set of sensor data generated by one or more sensors for a period of time;
generating at least two measures from the at least one set of sensor data;
determining a series of change point events for each measure for the period of time; and
determining a sleep stage for each interval of the period of time from at least two sleep stages by processing the series of change point events for each measure using a sleep stage classifier;
wherein the sleep stage classifier includes a set of parameters for each measure, the set of parameters for each measure including one or more coupling parameters, each coupling parameter being related to the cross-correlation between the each measure and another one of the measures.

12. The system according to claim 11, wherein the one or more sensors includes a photoplethysmographic (PPG) sensor and an accelerometer.

13. The system according to claim 12, wherein:
the at least two measures include actigraphy, tilt angle, and heart rate;
the determining the at least two measures includes:
determining the heart rate from the sensor data from the PPG sensor; and
determining the actigraphy and the tilt angle from the sensor data from the accelerometer.

14. The system according to claim 11, wherein the one or more sleep stages includes a sleep stage and a wake stage.

15. The system according to claim 11, wherein the set of parameters for each measure includes a sleep stage change event parameter and a history parameter.

16. The system according to claim 11, wherein:
the measures includes three measures; and
the set of parameters for each measure includes two coupling parameters.

17. The system according to claim 11, wherein the determining one or more sleep stages for each interval of the period of time includes:
applying the set of parameters for each measure to respective series of change point events to determine a probability of a change event; and
determining a probability of a change event for each interval of the period of time using each probability for each measure.

18. The system according to claim 17, wherein the determining one or more sleep stages for each interval of the period of time includes:
determining a sleep stage likelihood for each interval using the probability of the change event for each interval of time of the period of time; and
determining the sleep stage for each interval of time of the period of time from the sleep stage likelihood.

19. The system according to claim 11, wherein the non-transitory machine readable storage medium stores additional computer-executable instructions to cause the one or more processors to:
determining sleep information using the sleep stage for each interval of the period of time.

20. The system of according to claim 11, wherein the one or more processors and the non-transitory machine-readable storage medium are located in the wearable electronic device.
